(19) United States
(12) Patent Application Publication
(10) Pub. No.: US 2017/0360880 A1
(43) Pub. Date: Dec. 21, 2017

(54) ANTI-AIDS COCKTAIL CONSISTING OF AN ANTI-TUMORAL COMPOUND, A P-GLYCOPROTEIN INHIBITOR, AND AN ANTI-VIRAL AGENT

(71) Applicant: ARION TECNOLOGIA BRAZIL - GESTÄO DE ATIVOS S/A, CEP-60150-161 Fortaleza, CE (BR)

(72) Inventor: Gerson SILVA PAIVA, Rio de Janeiro (BR)

(73) Assignee: ARION TECNOLOGIA BRAZIL - GESTÄO DE ATIVOS S/A, CEP-60150-161 Fortaleza, CE (BR)

(21) Appl. No.: 15/538,140

(22) PCT Filed: Dec. 22, 2015
(86) PCT No.: PCT/BR2015/000196

§ 371 Date: Jun. 20, 2017

(30) Foreign Application Priority Data
Dec. 22, 2014 (BR) BR1020140331379
Jan. 5, 2015 (BR) BR1020150001150

(51) Int. Cl. A61K 38/12; A61K 39/12; G01N 33/567; C12Q 1/00

(52) U.S. Cl. CPC A61K 38/12; G01N 33/567; A61K 39/12; C12Q 1/00

(57) ABSTRACT

The invention relates to a cocktail against Aids, consisting of a commercially available anti-tumoral drug (1), such as doxorubicin, idarubicin, etoposide, chlorambucil, cisplatin, melphalan or bortezomib, a P-glycoprotein inhibitor (2), such as tariquidar, cetoconazol, verapamil, amiodarone or quinidine, and a commercially available antiviral agent of the viricidal type (3) which acts directly on the virus, such as N,N-dichloro-2,2-dimethyltaurine (NVC-422) or the HIVcide nanoviricide, and designed to combat and completely eliminate the Aids virus from the organism.

The present invention relates to an anti-Aids cocktail consisting of an anti-tumoral compound, a p-glycoprotein inhibitor and an anti-viral agent and in particular to a cocktail capable of completely eliminating an Aids virus from the body. The present invention makes use of the action of an anti-neoplastic agent for lowering to zero the number of lymphocytes in blood, more precisely the the TCD4+ lymphocytes, which constitute the hiding-place of HIV virus, the replication target of this virus in the human organism, and in turn become an anti-viral agent (virocide), which is the second component of the cocktail according to the present invention, efficient in the elimination of the virus from the organism, the inhibitor of the glycoprotein P acting in a potentializing way on the action of the anti-tumoral agent in the decrease of the number of lymphocytes in the blood.

In a further aspect, the invention relates to a cocktail against Aids, consisting of a commercially available anti-tumoral drug (1), such as doxorubicin, idarubicin, etoposide, chlorambucil, cisplatin, melphalan or bortezomib, a P-glycoprotein inhibitor (2), such as tariquidar, cetoconazol, verapamil, amiodarone or quinidine, and a commercially available antiviral agent of the viricidal type (3) which acts directly on the virus, such as N,N-dichloro-2,2-dimethyltaurine (NVC-422) or the HIVcide nanoviricide, and designed to combat and completely eliminate the Aids virus from the organism.

Aids (Acquired immunodeficiency syndrome) is a disease which is induced by an infection of the human organism by HIV (Human immunodeficiency virus). The HIV virus is of the retrovirus family and is responsible for Aids. This designation comprises at least two virus sub-categories, namely HIV-1 and HIV-2. In the HIV-1 exists a large variety of subtypes designated from -A to -J. When within the body, the virus infects primarily an important cell of immunologic system, referred to as the TCD4+ lymphocyte (or simply the lymphocyte T4), thereby reproducing itself and proliferating within the organism, resulting in the dreaded immunologic deficiency and in the symptoms of Aids, such as infections by funghi, bacteria (tuberculose) and other opportunistic viral, such as flue.

There is still no cure against Aids. The treatment of soro-positives is made on the basis of AZT or a cocktail presently composed of 19 remedies (every patient takes at least three), which together avoid the weakening of the immunologic system caused by the actuation of HIV. Its actuation diminishes the emanation of infections by opportunistic diseases (those which take advantage of a debilitated defence system). However, all medicines presently available against HIV, despite the fact of being capable of suppressing HIV infections to levels which can hardly be detected, these medicaments do not succeed in eradicating the virus completely from the organism, because the HIV may be hidden within the TCD4+ cells, and in this way replicate and proliferate within the organism. According to the present invention, a cocktail is used which contains a commercial anti-tumoral medicament, such as doxorubicin, or idarubicin, etoposide, chlorambucil, cisplatin, melflam, or bortezomib, or cyclosporine, and an inhibitor of P-glycoprotein, such as tariquidar, or cetoconazol, verapamil, miodaron, or quinidine, and an antiviral agent of the viricide type, which acts by destroying the virus outside the cells, thereby acting directly on the virus, such as N,N-dichloro-2,2-dimethyltaurin (NVC-422) or Nanoviricide (HIVCide).

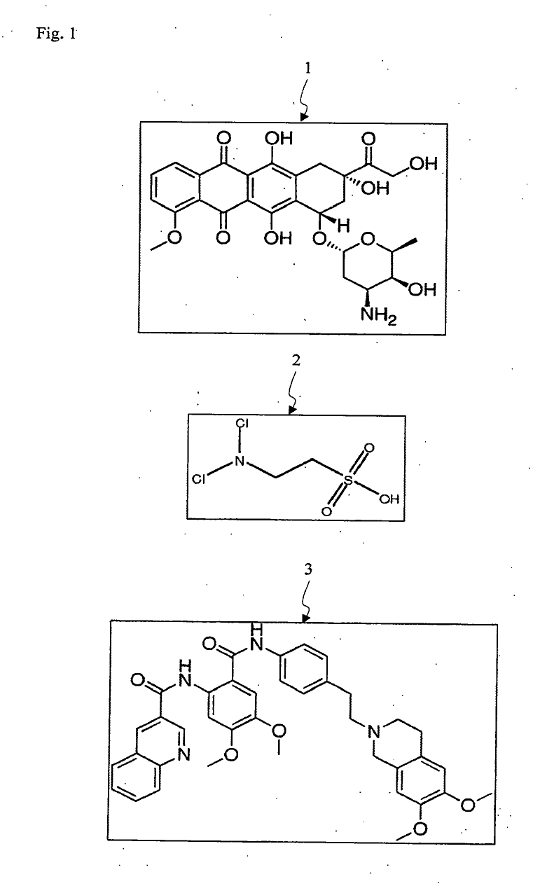

Referring now to the attached FIG. 1, this shows merely one composition possibility according to the inventive anti-Aids cocktail, formed by the combination of the antiviral agent N,N-dichloro-2,2-dimethyltaurin, with the anti-tumoral agent doxorubicin and with the inhibitor of the P-glycoprotein in this case tariquidar.

In accordance with the attached figure, the cocktail according to the present invention is an association of a commercial anti-tumoral medicament, such as doxorubicin (1), whose function is to destroy the hiding-place of the vira (i.e., the lymphocytes) by lowering the count of lymphocytes to zero in the blood by acting as an immunosuppressor, an inhibitor of P-glycoprotein, such as tariquidar (2), whose purpose is to maximize the preceding anti-tumoral compound in the lymphocytes, and an anti-virus of the viricide type, such as N,N-dichloro-2,2-dimethyltaurin (NVC-422) (3), which acts directly on the virus, whose purpose is to eliminate completely the Aids virus in the organism. The viricide agent NVC-422 oxidizes several amino-acids of the virus containing sulphur. The oxidation of these amino-acids results in alterations in the conformation of proteins and the morphology of the virus particle, resulting in a rapid loss of viral stability, deactivating them forever. In this way, the present anti-Aids cocktail will primarily lower to zero the number of lymphocytes in the blood of the infected individual, in view of the fact that the anti-tumoral agents above have a recognized immunosuppressive activity. In this way, the HIV virus is exposed in the blood plasma so that the anti-viral agent can act on them, deactivating them forever.

The concentration of the active agents (together) may vary considerably, but preferably is within the range 20 to 200 μg/ml of the commercial product, already including the other components, such as a vehicle.

Referring to said vehicles, these may be present in the form of diluents, as is well known in the state of the art. If diluents, carriers and/or other conventional additives are used, such as stabilizers, humidifying agents, emulsifiers, flavouring agents, buffers and similar, at least 1 to 80% by weight, preferably 1 to 25% by weight, and still more preferred, 2 to 15% by weight of the cocktail according to the invention should be composed of the three ingredients.

1. An anti-Aids cocktail comprising an anti-tumoral compound, a p-glycoprotein inhibitor and an anti-viral agent, characterized in that it is a cocktail comprising the combination of an anti-tumoral medicament, an inhibitor of glycoprotein P, and an antiviral agent of the type viricide, all known commercially and administered M their recommended doses.

2. Cocktail according to claim 1, characterized in that it eliminates completely the HIV virus in the organism by the destroying action of the lymphocytes TCD4+ (the hiding-place of the HIV virus) by the anti-tumoral compound and by the inhibitor of glycoprotein P, exposing the virus later to the direct action of the viricide agent, thereby eliminating completely all the vira in the organism.

3. Cocktail according to claim 1, characterized in that it is a cocktail capable of eliminating completely the HIV virus in the organism by the synergistic effect of an anti-tumoral pharmaceutical of commercial use (1), such as doxorubicin or idarubicin, etoposide, chlorambucil, cisplatin, melflam, or bortezomib, or cyclosporine, and an inhibitor of P-glycoprotein (2), such as tariquidar, or cetoconazol, verapamil, miodaron, or quinidine, and an antiviral agent of the viricide type (3), which acts directly on the virus, such as N,N-dichloro-2,2-dimethyltaurin (NVC-422) or Nanoviricide (HIVCide), whereby the anti-tumoral and the inhibitor of glycoprotein P will destroy the hiding-place of the HIV virus in the TCD4+ lymphocytes, exposing the virus to the direct action by one of the antiviral compounds of the viricide type as mentioned above.

4. Cocktail according to claim 2, characterized in that it is a cocktail capable of eliminating completely the HIV virus in the organism by the synergistic effect of an anti-tumoral pharmaceutical of commercial use (1), such as doxorubicin, or idarubicin, etoptside, chlorambucil, cisplatin, metflam, or bortezomib, or cyclosporine and a inhibitor of P-glycoprotein (2), such as tariquidar, or cetoconazol, verapamil, miodaron, or quinidine, and an antiviral agent of the viricide type (3), which acts directly on the virus, such as N,N-dichloro-2,2-dimethyltaurin (NVC-422) or Nanoviricide (HIVCide), whereby the anti-tumoral and the inhibitor of glycoprotein P will destroy the hiding-place of the HIV virus in the TCD4+ lymphocytes, exposing the virus to the direct action by one of the antiviral compounds the viricide type as mentioned above.
